(19) United States
(12) Patent Application Publication
(10) Pub. No.: US 2014/0276405 A1
(43) Pub. Date: Sep. 18, 2014

(54) Drug Delivery with an Expandable Polymeric Component

(71) Applicants: Christopher Robert Miller, Dulwich Hill (AU); Frank Risi, Dulwich Hill (AU); Ya Lang Enke, Baulkham Hills (AU)

(72) Inventors: Christopher Robert Miller, Dulwich Hill (AU); Frank Risi, Dulwich Hill (AU); Ya Lang Enke, Baulkham Hills (AU)

(21) Appl. No.: 14/199,097

(22) Filed: Mar. 6, 2014

Related U.S. Application Data
(60) Provisional application No. 61/787,669, filed on Mar. 15, 2013.

(51) Int. Cl. A61L 27/54; B29C 70/84; A61M 31/00

(52) U.S. Cl. CPC A61L 27/54; A61M 31/00; B29C 70/845
USPC 604/103.02; 264/255; 264/251

(57) ABSTRACT

Embodiments are generally directed to the delivery of drugs through the use of an expandable polymeric component that is configured to swell when exposed to a recipient's bodily fluid. More specifically, in one embodiment an apparatus for implantation into a recipient comprises an expandable polymeric component and a drug delivery element that releasably carries a drug. The drug delivery element at least partially surrounds the expandable polymeric component.

### CROSS-REFERENCE TO RELATED APPLICATIONS

The present application claims priority to U.S. Provisional Patent Application No. 61/787,669, filed on Mar. 15, 2013, the content of which is hereby incorporated by reference herein.

### BACKGROUND

1. Field of the Invention

The present invention relates generally to drug delivery in connection with an implantable medical device, and more particularly, to drug delivery with an expandable polymeric component.

2. Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, auditory brain implants, cochlear implants, etc.), implantable pacemakers, defibrillators, functional electrical stimulation devices, and other implantable medical devices, have been successful in performing life saving and/or lifestyle enhancement functions for a number of years.

Traditionally, there has been interest in delivering bioactive substances or chemicals (generally and collectively referred to herein as “drugs”) in conjunction with such implantable medical devices. Drugs may be delivered for a variety of purposes including, for example, to prevent infection and to facilitate healing implantation of the medical device.

### SUMMARY

In one aspect of the invention, an apparatus for implantation into a recipient is provided. The apparatus comprises a drug delivery element releasably carrying a drug; and an expandable polymeric component positioned adjacent to the drug delivery element such that the drug delivery element at least partially surrounds the expandable polymeric component.

In another aspect of the present invention, a method is provided. The method comprises

molding an expandable polymeric component configured to swell when exposed to the recipient's bodily fluid, and molding a polymeric drug delivery element loaded with a drug at least partially around the expandable polymeric component.

### BRIEF DESCRIPTION OF THE DRAWINGS

Embodiments of the present invention are described herein in conjunction with the accompanying drawings, in which:

FIG. 1 is a schematic diagram of a cochlear implant in which drug delivery techniques according to embodiments presented herein may be implemented;

FIG. 2 is a side view of an implantable component of a cochlear implant in which drug delivery techniques according to embodiments presented herein may be implemented;

FIG. 3A is a perspective view of a stimulating assembly that includes a drug delivery region in accordance with embodiments presented herein;

FIG. 3B is a cross-sectional view of the drug delivery region of FIG. 3A along line A-A;

FIG. 3C is a different cross-sectional view of the drug delivery region of FIG. 3A along line B-B;

FIG. 3D is a cross-sectional view of another drug delivery region in accordance with embodiments of the present invention;

FIG. 3E is a cross-sectional view of an alternative drug delivery region in accordance with embodiments of the present invention;

FIG. 4A is a perspective view of a stimulating assembly that includes a drug delivery region in accordance with embodiments presented herein;

FIG. 4B is a cross-sectional view of the drug delivery region of FIG. 4A along line A-A;

FIG. 4C is a different cross-sectional view of the drug delivery region of FIG. 4A along line B-B;

FIG. 5A is a perspective view of a stimulating assembly that includes a drug delivery region in accordance with embodiments presented herein;

FIG. 5B is a cross-sectional view of the drug delivery region of FIG. 5A along line A-A;

FIG. 5C is a different cross-sectional view of the drug delivery region of FIG. 5A along line B-B;

FIG. 6A is a perspective view of a stimulating assembly and a drug cover in accordance with embodiments presented herein;

FIG. 6B is a cross-sectional view of the stimulating assembly and drug cover of FIG. 6A; and

FIG. 7 is a flowchart of a method in accordance with embodiments presented herein.

### DETAILED DESCRIPTION

Embodiments are generally directed to the delivery of drugs through the use of an expandable polymeric component that is configured to swell when exposed to a recipient's bodily fluid. More specifically, in one embodiment an apparatus for implantation into a recipient comprises an expandable (swellable) polymeric component and a drug delivery element that releasably carries a drug.

For ease of illustration, the drug delivery techniques presented herein are primarily described in connection with a stimulating assembly of a cochlear implant (also commonly referred to as cochlear implant device, cochlear prosthesis, and the like; simply “cochlear implant” herein). However, it is to be appreciated that the drug delivery techniques may be used in conjunction with different implantable medical devices including other hearing prostheses (e.g., auditory brain stimulators, mechanical stimulators, etc.), sensors, implantable pacemakers, defibrillators, functional electrical stimulation devices, catheters, etc.

FIG. 1 is perspective view of an exemplary cochlear implant 100 that comprises an external component 142 and an internal or implantable component 144. The external component 142 is directly or indirectly attached to the body of the recipient and typically comprises one or more sound input elements 124 (e.g., microphones, telecoils, etc.) for detecting sound, a sound processor 126, a power source (not shown), an external coil 130 and, generally, a magnet (not shown) fixed relative to the external coil 130. The sound processor 126 processes electrical signals generated by a sound input element 124 that is positioned, in the depicted embodiment, by auricle 110 of the recipient. The sound processor 126 provides the processed signals to external coil 130 via a cable (not shown).

The internal component 144 comprises an elongate stimulating assembly 118, a stimulator unit 120, and an internal receiver/transceiver unit 132, sometimes referred to herein as transceiver unit 132. The transceiver unit 132 is connected to an internal coil 136 and, generally, a magnet (not shown) fixed relative to the internal coil 136. Internal transceiver unit 132 and stimulator unit 120 are sometimes collectively referred to herein as a stimulator/transceiver unit.

The magnets in the external component 142 and internal component 144 facilitate the operational alignment of the external coil 130 with the internal coil 136. The operational alignment of the coils enables the internal coil 136 to transmit/receive power and data to/from the external coil 130. More specifically, in certain examples, external coil 130 transmits electrical signals (e.g., power and stimulation data) to internal coil 136 via a radio frequency (RF) link. Internal coil 136 is typically a wire antenna coil comprised of multiple turns of electrically insulated single-strand or multi-strand platinum or gold wire. The electrical insulation of internal coil 136 is provided by a flexible silicone molding. In use, transceiver unit 132 may be positioned in a recess of the temporal bone of the recipient. Various other types of energy transfer, such as infrared (IR), electromagnetic, capacitive and inductive transfer, may be used to transfer the power and/or data from an external device to cochlear implant and FIG. 1 illustrates only one example arrangement.

Elongate stimulating assembly 118 is implanted in cochlea 140 and includes a contact array 146 comprising a plurality of stimulating contacts 148. Stimulating assembly 118 extends through cochleostomy 122 and has a proximal end connected to stimulator unit 120 via lead region 108 that extends through mastoid bone 119.

FIG. 2 is a simplified side view of an internal component 244 having a stimulator/receiver unit 202 which receives encoded signals from an external component of the cochlear implant system. Internal component 244 terminates in a stimulating assembly 218 that comprises an extra-cochlear region 210 and an intracochlear region 212. Intra-cochlear region 212 is configured to be implanted in the recipient's cochlea and has disposed thereon a contact array 216. In the present example, contact array 216 comprises both optical stimulating contacts 220 and electrical stimulating contacts 230. Present commercial devices offered by the industry use electrical contacts, but Cochlear and others are engaged in research on the potential uses of optical stimulation alone or in conjunction with electrical or other stimulation mechanisms.

There are a variety of types of intra-cochlear stimulating assemblies including short, straight and perimodiolar. A perimodiolar stimulating assembly 218 is configured to adopt a curved configuration during and or after implantation into the recipient's cochlea. To achieve this, in certain arrangements, stimulating assembly 218 is pre-curved to the same general curvature of a cochlea. In such examples, stimulating assembly 218 is typically held straight by a stiffening stylet or a sheath which is removed during implantation, or alternatively by varying material combinations or the use of shape memory materials so that the stimulating assembly may adopt its curved configuration when in the cochlea. Other methods of implantation, as well as other stimulating assemblies which adopt a curved configuration, may also be used.

Stimulating assembly 218 can also be a non-perimodiolar stimulating assembly. For example, stimulating assembly 218 may comprise a straight stimulating assembly or a mid-scala assembly which assumes a midscale position during or following implantation.

Alternatively, the stimulating assembly may be a short electrode implanted into at least a basal region. The stimulating assembly may extend towards an apical end of the cochlea, referred to as the cochlea apex. In certain circumstances, the stimulating assembly may be inserted into the cochlea via a cochleostomy. In other circumstances, a cochleostomy may be formed through the round window, the oval window, the promontory or through an apical turn of cochlea.

Internal component 244 further comprises a lead region 208 coupling stimulator/receiver unit 202 to stimulating assembly 218. Lead region 208 comprises a region 204 which is commonly referred to as a helix region, however, the required property is that the lead accommodate movement and is flexible, it does not need to be formed from wire wound helically. Lead region also comprises a transition region 206 which connects helix region 204 to stimulating assembly 218. As described below, optical and/or electrical stimulation signals generated by stimulator/receiver unit 202 are delivered to contact array 216 via lead region 308. Helix region 204 prevents lead region 208 and its connection to stimulator/receiver 202 and stimulating assembly 218 from being damaged due to movement of internal component 244 (or part of 244) which may occur, for example, during mastication.

Many implantable medical devices, such as cochlear implants, employ components that are intended to remain implanted in a recipient for an extended period of time (e.g., permanently). As such, to promote proper and “healthy” implantation, there have been a number of proposals for delivering drugs to the implant site along with an implantable component such as an intra-cochlear stimulating assembly. These proposals include, for example, loading drugs into the polymeric body of the stimulating assembly so that the drugs passively escape or “elute” from the polymeric material after implantation.

Successful delivery of drugs to an implant site can provide benefits that include, for example, faster recovery following the implantation trauma, infection/disease prevention, an increase in stimulation effectiveness (e.g., by supporting hair cell survival and growth in cochlear implants), directly targeting diseases such as tinnitus, promoting acceptance of the implant at the site, and facilitating the function of the implant. As used herein, the term “drug” includes, but is not limited to, bioactive substances or chemicals used for therapeutic, prophylactic, and/or diagnostic purposes, including active pharmaceutical ingredients (APIs) (e.g., anti-inflammatories, anti-microbials, fibrotics, etc.).

FIG. 3A is a perspective view of a stimulating assembly 318 in accordance with embodiments presented herein. For ease of illustration, only a portion of the stimulating assembly 318 is shown in FIG. 3A.

Stimulating assembly 318 comprises a carrier member 350 that includes a contact array 316. Contact array 316 includes a plurality of contacts 330 longitudinally spaced along carrier member 350. A portion of carrier member 350 is formed as a drug delivery region 360 that is described further below.

FIG. 3B is a cross-sectional view of drug delivery region 360 through line A-A of FIG. 3A, while FIG. 3C is a cross-sectional view of drug delivery region 360 through line B-B of FIG. 3A and is shown separate from the remainder of carrier member 350. The remainder of the carrier member 350 is sometimes referred to herein as the main body of the carrier member. As shown, drug delivery region 360 has a generally cylindrical shape.

In the embodiments of FIGS. 3A-3C, drug delivery region 360 comprises a substantially cylindrical elongate core 362 formed from a polymeric material that is configured to swell (i.e., expand) when exposed to a recipient's bodily fluid. As such, core 362 is sometimes referred to herein as an expandable polymeric component or expandable core.

Core 362 is substantially surrounded by a drug delivery layer 364 that is loaded (doped) with one or more drugs. In FIG. 3B, drug delivery layer 364 is an annular shaped member that releasably carries one or more drugs that are configured to elute from drug delivery layer during or after implantation into a recipient. Drug delivery layer 364 is sometimes referred to herein as a drug delivery element. Although drug delivery layer 364 is loaded with one or more drugs, core 362 is not loaded with drugs and remains substantially drug-free, except for possible minor amounts of drugs that may migrate or leach from drug delivery layer 364 into drug-free central core 362.

In specific embodiments of FIGS. 3A-3C, core 362 and drug delivery layer 364 are each formed from polymeric materials. Core 362 may be formed from a number of different expandable polymeric materials such as, for example, Polydimethylsiloxane (PDMS). Drug delivery layer 364 may be formed from the same material, a different material, or a different grade of the same material as core 362. In one specific embodiment, core 362 and drug delivery layer 364 are each formed from PDMS elastomers. The main body of the carrier member 350 (i.e., the portions outside of the drug delivery region 360) may be formed from the same or different polymeric material(s) used in the drug delivery region or core.

In the embodiments of FIGS. 3A-3C, during or after implantation, the core 362 is exposed to the recipient's bodily fluid and accordingly swells and expands as shown by arrows 361. That is, the core 362 takes on the cochlea fluid (perilymph) (i.e., the cochlea fluid penetrates the matrix and the polymeric material swells). In one specific embodiment, the core 362 may expand by approximately 2 to approximately 3 percent. The expansion of the core 362 places a hoop strain/stress (i.e., a circumferentially exerted force) on the outer surface of the drug delivery layer 364. Additionally, because only the outer layer of the cylindrical shaped drug delivery region 360 is loaded with the drug, the drug has a short diffusion distance.

In the embodiments of FIGS. 3A-3C, core 362 has a diameter 363 and drug delivery layer 364 has a thickness 365. The diameter 363 relative to the thickness 365 may affect the drug elution kinetics. For example, a core 362 having a diameter that is substantially large relative to the thickness of the drug delivery layer 364 may cause faster drug elution than a core having a diameter that is the same size as, or smaller than, the thickness of the drug delivery layer. As such, the ratio of the diameter 363 to the thickness 365 may vary in different embodiments based on, for example, desired elution kinetics, polymeric materials, drug, drug loadings, etc.

As shown in FIGS. 3B and 3C, wires 367 from contacts 330 positioned distally to the drug delivery region 360 extend through the central core 362. In alternative embodiments, the wires 367 may extend through the drug delivery layer 364 or through a separate polymeric element (not shown).

FIGS. 3A-3C illustrate embodiments in which the core 362 is generally cylindrical with a circular cross-sectional shape. It is to be appreciated that cores having different shapes (e.g., elliptical, oval, rectangular etc.) may be used in other embodiments.

As noted, the core 362 swells when bodily fluid enters the polymeric matrix of the core. In certain embodiments, the bodily fluid may access the core 362 via the main body of the carrier member 350 (e.g., through the ends of the drug delivery region 360). FIG. 3D illustrates one alternative embodiment in which the drug delivery layer 364 includes access pathway to facilitate the flow of bodily fluid to the core 362.

More specifically, FIG. 3D illustrates a drug delivery region 360D that comprises an expandable core 362 (as described above) and a drug delivery layer 364D that is similar to the drug delivery layer 364 of FIGS. 3A-3C. However, in the specific example of FIG. 3D, drug delivery layer 364D includes a plurality of apertures 374 that provide direct access pathways for the cochlea fluid to flow into to core 362.

It is to be appreciated that the use of apertures in the drug delivery layer is only an example of access pathways that may be used in embodiments presented herein. For example,

FIG. 3E illustrates an alternative embodiment in which a dedicated lumen (through-hole) 381 runs lengthwise through a core 362E of a drug delivery region 360E. The lumen 381 enables communication of fluid from the environment surrounding the device to the core 362E. In certain embodiments, the lumen 381 may also be used for a stylet.

FIGS. 3A-3E illustrate embodiments in which a drug delivery region is configured to deliver drugs to a recipient through the use of an expandable polymeric component (core) and a drug delivery element (drug delivery layer). FIGS. 4A to 6B illustrate alternative embodiments for delivering drugs to a recipient through the use of expandable polymeric components and associated drug delivery elements.

More specifically, FIG. 4A is a perspective view of a portion of a stimulating assembly 418 in accordance with other embodiments presented herein. Stimulating assembly 418 comprises a carrier member 450 that includes a contact array 416. Contact array 416 includes a plurality of contacts 430 longitudinally spaced along carrier member 450. As described further below, the distal end or tip of carrier member 450 is a drug delivery region 460. Drug delivery region 460 is sometimes referred to herein as drug delivery tip 460. The portion of the carrier member 450 that is outside of the drug delivery tip 460 is sometimes referred to herein as the main body of the carrier member.

FIG. 4B is a cross-sectional view of drug delivery tip 460 through line A-A of FIG. 4A, while FIG. 4C is a cross-sectional view of drug delivery tip 460 through line B-B of FIG. 4A. As shown, drug delivery tip 460 has a generally conical shape.

In the embodiments of FIGS. 4A-4C, drug delivery tip 460 comprises a conical shaped core 462 formed from a polymeric material that is configured to swell (i.e., expand) when exposed to a recipient's bodily fluid. As such, core 462 is sometimes referred to herein as an expandable polymeric component.

Core 462 is substantially surrounded by a drug delivery layer 464 that is loaded with one or more drugs. That is, drug delivery layer 464 is a conical shaped member that releasably carries one or more drugs that are configured to elute from drug delivery layer during or after implantation into a recipient. Drug delivery layer 464 is sometimes referred to herein as a drug delivery element. Although drug delivery layer 364 is loaded with one or more drugs, core 462 is not loaded with drugs and remains substantially drug-free, except for possible minor amounts of drugs that may migrate or leach from drug delivery layer 464 into drug-free central core 462.

In specific embodiments of FIGS. 4A-4C, core 462 and drug delivery layer 464 are each formed from polymeric materials. Core 462 may be formed from a number of different expandable polymeric materials such as, for example, a PDMS elastomer. Drug delivery layer 464 may be formed from the same material, a different material, or a different grade of the same material as core 462. In one specific embodiment, central core 462 and drug delivery layer 464 are each formed from a PDMS elastomer. The main body of the carrier member 450 (i.e., the portions outside of the drug delivery tip 460) may be formed from the same or different polymeric material(s) used in the drug delivery tip.

In the embodiments of FIGS. 4A-4C, the core 462 is configured to increase the surface area of the drug deliver layer 464 so as to improve elution kinetics after the stimulating assembly 418 is implanted in a recipient.

More specifically, during or after implantation the core 462 is exposed to the recipient's bodily fluid and accordingly swells and expands outward as shown by arrows 461. That is, the core 462 takes on the cochlea fluid (i.e., the cochlea fluid penetrates the matrix and the polymeric material swells). In one specific embodiment, the core 462 may expand by approximately 2 to approximately 3 percent. The expansion of the core 462 places forces on the outer surface of the drug delivery layer 464 such that the surface area of the drug delivery layer 464 increases so as to induce rapid elution of the drug(s).

In certain embodiments, the bodily fluid may access the core 462 via the main body of the carrier member 450 (e.g., through the proximal of the drug delivery tip 460). In alternative embodiments, the drug delivery layer 464 may include access pathways (such as the apertures shown in FIG. 3D) that facilitate the flow of bodily fluid to the core 462.

In the embodiments of FIGS. 4A-4C, core 462 has a diameter that decreases towards the distal end of the drug delivery tip 460. The drug delivery layer 464 has a thickness that, as shown in FIG. 4C, also decreases towards the distal end of the drug delivery tip 460.

The diameter of the core 462 relative to the thickness of the drug delivery layer 464 at any point along the drug delivery tip 460 may affect the drug elution kinetics. For example, a core 462 having a diameter at a specific location that is substantially large relative to the thickness of the drug delivery layer 464 at the same location may cause faster drug elution than a core having a diameter at a specific location that is the same as, or smaller than, the thickness of the drug delivery layer at that specific location. As such, the diameter-to-thickness ratio at a point along the drug delivery tip 460 may vary in different embodiments based on, for example, one or more of the desired elution kinetics, desired delivery time frame, polymeric materials, drug, drug loadings, etc.

In certain embodiments, the thickness of the drug delivery layer 464 may decrease in proportion to the decrease in the diameter of the core 462 such that a substantially constant diameter-to-thickness ratio is maintained. In alternative embodiments, the drug delivery layer 464 has a substantially constant thickness even though the diameter of the core 462 decreases. Other combinations of diameters-to-thicknesses may be used in alternative embodiments.

FIGS. 4A-4C illustrate embodiments in which the core 462 has a conical shape. In alternative embodiments, the core 462 could have a cylindrical or other shape. Additionally, drug delivery tip 460 may integrated with the main body (remainder of carrier member 450) or a separate member that is attached to the main body of the carrier member via, for example, a permanent adhesive.

FIG. 5A is a perspective view of a portion of a stimulating assembly 518 in accordance with other embodiments presented herein. Stimulating assembly 518 comprises a carrier member 550 that includes a contact array 516. Contact array 516 includes a plurality of contacts 530 longitudinally spaced along carrier member 550. As described further below, a portion of the carrier member 550 is formed as a drug delivery region 560 that extends along a length of the carrier member. The portion of the carrier member 550 that is not part of the drug delivery region 560 is sometimes referred to herein as the main body of the carrier member.

FIG. 5B is a cross-sectional view of drug delivery region 560 through line A-A of FIG. 5A, while FIG. 5C is a cross-sectional view of a portion of drug delivery region 560 through line B-B of FIG. 5A.

In the embodiments of FIGS. 5A-5C, drug delivery region 560 comprises an elongate core 562 formed from a polymeric material that is configured to swell (i.e., expand) when exposed to a recipient's bodily fluid. As such, core 562 is sometimes referred to herein as an expandable polymeric component or expandable core. As shown in FIG. 5B, core 562 is a cylindrical member having a circular cross-sectional shape.

Core 562 is adjacent to, and partially surrounded by, a drug delivery layer 564 that is loaded with one or more drugs. As shown, drug delivery layer 564 is a generally arcuate member that abuts approximately half to approximately two-thirds or more of the core 562. The drug delivery layer 564 releasably carries one or more drugs that are configured to elute from drug delivery layer 564 during or after implantation into a recipient and, as such, is sometimes referred to herein as a drug delivery element. Although drug delivery layer 564 is loaded with one or more drugs, core 562 is not loaded with drugs and remains substantially drug-free, except for possible minor amounts of drugs that may migrate or leach from drug delivery layer 564 into drug-free central core 562.

As shown in FIG. 5B, carrier member 550 also comprises a polymeric main body 570. A surface 590 forming the ends of the arcuate shape of the drug delivery layer 564 abut the main body 570 so as to define a space or volume between a portion of the main body 570 and a portion of the drug delivery layer 564. The core 562 is disposed in this space between the main body 570 and the drug delivery layer 564. In other words, the core 562 is positioned between the arcuate drug delivery layer 564 and the main body 570.

In specific embodiments of FIGS. 5A-5C, core 562 and drug delivery layer 564 are each formed from polymeric materials. Core 562 may be formed from a number of different expandable polymeric materials such as, for example, a PDMS elastomer. Drug delivery layer 564 may be formed from the same material, a different material, or a different grade of the same material as core 562. In one specific embodiment, core 562 and drug delivery layer 664 are each formed from a PDMS elastomer. The main body 570 may be formed from the same or different polymeric material(s) used in the drug delivery region 560.

In the embodiments of FIGS. 5A-5C, the core 562 is configured to increase the surface area of the drug deliver layer 564 so as to improve elution kinetics after the stimulating assembly 518 is implanted in a recipient.

More specifically, during or after implantation, the core 562 is exposed to the recipient's bodily fluid and accordingly swells and expands outward in at least the directions shown by arrows 561. That is, the core 562 takes on the cochlea fluid (perilymph). As the cochlea fluid penetrates the matrix, the polymeric material swells. In one specific embodiment, the core 562 may expand by approximately 2 to approximately 3 percent. The expansion of the core 562 places forces on the outer surface of the drug delivery layer 564 such the surface area of the drug delivery layer 564 increases so as to induce rapid elution of the drug(s).

In certain embodiments, the bodily fluid may access the core 562 via the remainder of the carrier member 550 (e.g., through the main body 570). In alternative embodiments, the drug delivery layer 564 may include access pathways (such as the apertures shown in FIG. 3D) that facilitate the flow of bodily fluid to the core 562.

In the embodiments of FIGS. 5A-5C, core 562 has a diameter 563 and drug delivery layer 564 has a thickness 565. The diameter 563 relative to the thickness 565 may affect the drug elution kinetics. For example, a core 562 having a diameter that is substantially large relative to the thickness of the drug delivery layer 564 may cause faster drug elution than a central core having a diameter that is the same size as, or smaller than, the thickness of the drug delivery layer. As such, the ratio of the diameter 563 to the thickness 565 may vary in different embodiments based on, for example, one or more of the desired elution kinetics, polymeric materials, drug(s), drug loadings, etc.

As shown in FIG. 5B, wires 567 from contacts 530 extend through a main body 570 of carrier member 550. In alternative embodiments, the wires 567 may extend through the drug delivery layer 564 or through core 562.

FIGS. 5A-5C illustrate embodiments in which the core 462 is cylindrical with a circular cross-sectional shape. In alternative embodiments, the core 462 could have other shapes (e.g., oval, elliptical, or other cross-sectional shapes).

FIG. 6A is a perspective view of a stimulating assembly 618 in accordance with other embodiments presented herein. FIG. 6B is a cross-sectional view of stimulating assembly 618 and drug cover 660 drug through line A-A of FIG. 6A

Stimulating assembly 618 comprises a carrier member 650 that includes a contact array 616. Contact array 616 includes a plurality of contacts 630 longitudinally spaced along carrier member 650. Contacts 630 are each connected to a wire 637. As described further below, a drug cover 660 is disposed on a length of the carrier member 650.

In the embodiments of FIGS. 6A and 6B, drug cover 660 comprises a first arcuate layer 662 positioned abutting the outer surface of the main body of the carrier member 650. The layer 662 is configured to swell (i.e., expand) when exposed to a recipient's bodily fluid. As such, layer 662 is sometimes referred to herein as an expandable polymeric component or expandable layer.

Disposed on the outer surface of expandable layer 662 (i.e., the surface that is opposite to the carrier member 650) is a second arcuate layer 664. That is, expandable layer 662 is a layer disposed between the carrier member 650 and the second layer 664. The second layer 664 releasably carries one or more drugs that are configured to elute from the layer during or after implantation into a recipient. As such, the layer 664 is sometimes referred to herein as a drug delivery element or drug delivery layer. Although drug delivery layer 664 is loaded with one or more drugs, expandable layer 662 is not loaded with drugs and remains substantially drug-free, except for possible minor amounts of drugs that may migrate from drug delivery layer 664 into expandable layer 662.

In specific embodiments of FIGS. 6A and 6B, expandable layer 662 and drag delivery layer 664 are each formed from polymeric materials. Expandable layer 662 may be formed from a number of different expandable polymeric materials such as, for example, a PDMS elastomer. Drug delivery layer 664 may be formed from the same material, a different material, or a different grade of the same material as expandable layer 662. In one specific embodiment, expandable layer 662 and drug delivery layer 664 are each formed from a PDMS elastomer. Carrier member 650 may be formed from the same or different polymeric material(s) used in the drug cover 660.

Elution kinetics may be controlled by the surface area of the polymeric material. In the embodiments of FIGS. 6A and 6B, the expandable layer 662 is configured to increase the surface area of the drug deliver layer 664 so as to improve elution kinetics after the stimulating assembly 618 is implanted in a recipient.

More specifically, during or after implantation, the expandable layer 662 is exposed to the recipient's bodily fluid and accordingly swells and expands outward in at least the directions shown by arrows 661. That is, the expandable layer 662 takes on the cochlea fluid. In one specific embodiment, the expandable layer 662 may expand by approximately 2 to approximately 3 percent. The expansion of the expandable layer 662 places forces on the outer surface of the drug delivery layer 664 such that the surface area of the drug delivery layer 664 increases so as to induce rapid elution of the drug(s).

In certain embodiments, the bodily fluid may access the core 662 via the carrier member 650 and/or through the ends of the layer that are directly exposed to the fluid. In alternative embodiments, the drug delivery layer 664 may include access pathways (such as the apertures shown in FIG. 3D) that facilitate the flow of bodily fluid to the core 662.

In the embodiments of FIGS. 6A and 6B, expandable layer 662 has a thickness 663 while drug delivery layer 664 has a thickness 665. The thickness 663 relative to the thickness 665 of the drug delivery layer 664 may affect the drug elution kinetics. For example, an expandable layer 662 having a thickness that is substantially large relative to the thickness of the drug delivery layer 664 may cause faster drug elution than an expandable layer having a diameter that is the same size as, or smaller than, the thickness of the drug delivery layer. As such, the ratio of the thickness 663 to the thickness 665 may vary in different embodiments based on, for example, one or more of the desired elution kinetics, polymeric materials, drug(s), drug loadings, etc.

In certain embodiments, drug cover 660 may be integrated with the carrier member 650. Alternatively, drug cover 660 may be a separate member that is attached to the carrier member 650 via, for example, a permanent adhesive.

FIGS. 3A to 6B illustrate various embodiments of the present invention that are merely illustrative. It is to be appreciated that various modifications to the illustrated embodiments may be made and that the various embodiments are not mutually exclusive. For example, in certain circumstances, a stimulating assembly may include multiple drug delivery regions as described above with reference to FIGS. 3A-3C and 4A-4C. Alternatively, one or more multiple drug delivery regions as described above with reference to FIGS. 3A-3 may be used with the drug delivery tip of FIGS. 5A-5C. These are merely illustrative example combinations and other combinations are possible.

FIG. 7 is a flowchart of a method 700 in accordance with embodiments presented herein. Method 700 begins at block 702 which molding an expandable polymeric component configured to swell when exposed to the recipient's bodily fluid. At block 704, a polymeric drug delivery element loaded with a drug is molded at least partially around the expandable polymeric component.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. An apparatus for implantation into a recipient, comprising:
a drug delivery element releasably carrying a drug; and
an expandable polymeric component positioned adjacent to the drug delivery element such that the drug delivery element at least partially surrounds the expandable polymeric component,
wherein the expandable polymeric component is configured to swell when exposed to the recipient's bodily fluid.

2. The apparatus of claim 1, wherein the expandable polymeric component is an elongate expandable polymeric component and wherein the drug delivery element substantially surrounds an outer surface of the elongate expandable polymeric component.

3. The apparatus of claim 2, further comprising:
an elongate main body,
wherein the elongate expandable polymeric component and the drug delivery element are disposed at a distal end of the elongate main body.

4. The apparatus of claim 3, wherein the elongate expandable polymeric component has a generally conical shape.

5. The apparatus of claim 1, further comprising:
an elongate main body,
wherein the drug delivery element has an arcuate first surface and a second surface positioned abutting the main body so as to define a space between the main body and the drug delivery element, and
wherein the expandable polymeric component comprises a substantially cylindrical elongate element disposed in the space between the main body and the drug delivery element.

6. The apparatus of claim 1, further comprising:
an elongate main body,
wherein the expandable polymeric component comprises an arcuate layer disposed on a portion of an outer surface of the main body, and wherein the drug delivery element comprises an acruate layer disposed on the expandable polymeric component.

7. The apparatus of claim 1, wherein the expandable polymeric component is a substantially drug-free element.

8. The apparatus of claim 1, wherein the expandable polymeric component is a Polydimethylsiloxane (PDMS) element.

9. The apparatus of claim 1, wherein the expandable polymeric component and the drug delivery element are formed from the same material.

10. The apparatus of claim 1, wherein the expandable polymeric component and the drug delivery element are formed from different materials.

11. The apparatus of claim 1, wherein the drug delivery element includes access pathways to facilitate flow of the bodily fluid to the expandable polymeric component.

12. A method, comprising:
molding an expandable polymeric component configured to swell when exposed to the recipient's bodily fluid; and
molding a polymeric drug delivery element loaded with a drug at least partially around the expandable polymeric component.

13. The method of claim 12, further comprising:
molding a polymeric elongate main body; and
attaching the elongate expandable polymeric component and the drug delivery element to a distal end of the elongate main body.

14. The method of claim 13, further comprising:
molding the expandable polymeric component into a generally conical shape.

15. The method of claim 12, further comprising:
molding a polymeric elongate main body;
molding the drug delivery element into a generally arcuate shape having a surface positioned abutting the main body so as to define a space between the main body and the drug delivery element, and
molding the expandable polymeric component as a substantially cylindrical elongate element disposed in the space between the main body and the drug delivery element.

16. The method of claim 12, further comprising:
molding a polymeric elongate main body;
molding the expandable polymeric component as an arcuate layer disposed on a portion of an outer surface of the main body; and
molding the drug delivery element as an acruate layer disposed on the expandable polymeric component.

17. The method of claim 12, wherein molding the expandable polymeric component molding a substantially drug-free element expandable polymeric component.

18. The method of claim 12, wherein molding the expandable polymeric component molding a Polydimethylsiloxane (PDMS) element.

19. The method of claim 12, further comprising:
molding the expandable polymeric component and the drug delivery element from the different materials.

20. The method of claim 12, further comprising:
molding the drug delivery element to include access pathways to facilitate flow of the bodily fluid to the expandable polymeric component.
